(19) United States
(12) Patent Application Publication
(10) Pub. No.: US 2014/0200233 A1
(43) Pub. Date: Jul. 17, 2014

(54) USE OF MAD2L2 AS A STRATIFICATION MARKER IN THE TREATMENT OF BREAST TUMOURS WITH NOVEL PAN-CDK INHIBITORS

(71) Applicants: Gerhard Siemeister, Berlin (DE); Philip Groth, Berlin (DE)

(72) Inventors: Gerhard Siemeister, Berlin (DE); Philip Groth, Berlin (DE)

(73) Assignee: BAYER INTELLECTUAL PROPERTY GMBH, Monheim (DE)

(21) Appl. No.: 14/239,137

(22) PCT Filed: Aug. 15, 2012
(86) PCT No.: PCT/EP2012/065944

§ 371 Date: Feb. 15, 2014

(30) Foreign Application Priority Data
Aug. 16, 2011 (DE) 10 2011 080 992.9

(51) Int. Cl. C07D 239/47

(52) U.S. Cl. CPC C07D 239/47
USPC 514/272; 435/29; 506/9

(57) ABSTRACT

The invention relates to the use of MAD2L2 as stratification marker in the treatment of breast tumours with novel pan-CDK inhibitors of the formula (I).

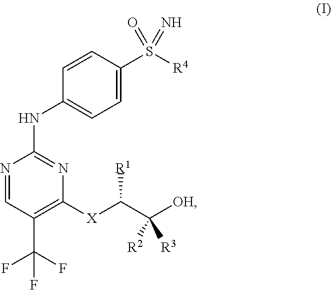

The invention relates to the use of MAD2L2 as stratification marker in the treatment of breast tumours with novel pan-CDK inhibitors.

The eukaryotic cell division cycle ensures duplication of the genome and its distribution to the daughter cells by passing through a coordinated and regulated sequence of events. The cell cycle is divided into four successive phases: the G1 phase represents the time prior to DNA replication in which the cell grows. In the S phase, the cell replicates its DNA, and in the G2 phase it prepares for entering mitosis. During mitosis (M phase), the replicated DNA is separated and the cell division is performed. Cyclin-dependent kinases (CDKs), a family of serine/threonine kinases whose members require binding of a cyclin (Cyc) as regulatory subunit for their activation, drive the cell through the cell cycle. Various CDK/Cyc pairs are active in the different phases of the cell cycle. CDK/Cyc pairs important for the basic function of the cell cycle are, for example, CDK4(6)/CycD, CDK2/CycE, CDK2/CycA, CDK1/CycA and CDK1/CycB. Thus, for example, the activities of the CDK4(6)/CycD and CDK2/CycE complexes drive the entry of a cell into the cell cycle and the passing of the “restriction point”, which marks the independence of a cell from further growth signals for the end of the cell division initiated.

A number of control mechanisms ensure the ordered progression of the cell division phases and the correct division of the duplicated genetic material to the daughter cells. Inter alia, the activity of the CDKs is affected by inhibitory proteins such as, for example, p21, p16 or p27, and the expression and the degradation of the cyclins is regulated. During the mitosis phase of the cell division cycle, the proteins of the spindle assembly checkpoint ensure correct adhesion of the spindle apparatus to the duplicated chromosomes and correct distribution of the chromosomes to the daughter cells. Essential proteins of the spindle assembly checkpoint are MAD1, MAD2, BUB1, BUBR1, TTK (Mps-1) and cdc20. In human cells, there are two isoforms of the MAD2 protein, MAD2L1 and MAD2L2 (MAD2B).

A deregulated expression of cyclin E and the occurrence of cyclin E fragments result in overactivation of the CDK2/CycE complex and stimulation of the cell division cycle, which lead to the hypothesis that patients having tumoral cyclin E overexpression might, with higher probability, benefit from a CDK2-directed therapy (Hunt, K. K., Keyomarsi, K., Sem. Cancer Biol. 15, 319, 2005).

Rimkus et al (Int. J. Cancer 120, 207, 2006) describe an at least 3-fold elevated expression of MAD2L2 in 25 of 118 (21%) human colon carcinoma samples examined. The elevated expression of MAD2L2 correlated with a reduced survival time of the patients.

Although CDK inhibitors have been in clinical development for more than 10 years, hitherto, no biomarkers have been described which allow prediction of the response of a patient to the therapy with CDK inhibitors. Such stratification markers allow the targeted therapy of those patients who would, with high probability, benefit from a CDK inhibitor therapy. Moreover, stratification markers increase the probability of success of clinical studies.

WO2010/046035 discloses particularly effective pan-CDK inhibitors of the formula (I)

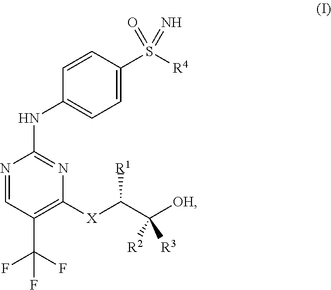

in which
X represents —O— or —NH—, and
R¹ represents a methyl, ethyl, propyl or isopropyl group, and
R² and R³ independently of one another represent hydrogen, a methyl or ethyl group, and
R⁴ represents a C₁-C₆-alkyl group or a C₃-C₇-cycloalkyl ring,
and their salts, diastereomers and enantiomers.

The application is based on the following definitions:

### C₁-C₆-Alkyl

A C₁-C₆-alkyl group is understood to mean in each case a straight-chain or branched alkyl radical such as, for example, a methyl, ethyl, propyl, isopropyl, butyl, isobutyl, sec-butyl, tert-butyl, pentyl, isopentyl or a hexyl radical.

C₃-C₇-Cyclo alkyl

A C₃-C₇-cycloalkyl ring is understood to mean a cyclopropyl, cyclobutyl, cyclopentyl, cyclohexyl or a cycloheptyl ring.

In the general formula (I), X may represent —O— or —NH—.

Preferably, X represents —O—.

In the general formula (I), R¹ may represent a methyl, ethyl, propyl or isopropyl group.

Preferably, R¹ represents a methyl group.

In the general formula (I), R² and R³ independently of one another may represent hydrogen, a methyl or ethyl group.

Preferably, R² and R³ independently of one another represent hydrogen or a methyl group.

Particularly preferably, R² represents a methyl group and R³ represents hydrogen or a methyl group.

In the general formula (I), R⁴ may represent a C₁-C₆-alkyl radical or a C₃-C₇-cycloalkyl ring.

Preferably, R⁴ represents a methyl or ethyl group or represents a cyclopropyl ring.

Of particular interest is the compound (2R,3R)-3-{[2-{[4-(R-cyclopropylsulphonimidoyl)phenyl]amino}-5-(trifluoromethyl)pyrimidin-4-yl]oxy}butan-2-ol (Compound A).

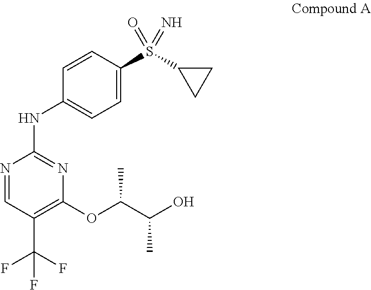

The diastereomers of the formula I were separated by preparative chromatography. The experimental details are given in WO2010/046035A1.

It is an object of the present invention to provide a stratification marker for the pan-CDK inhibitors of WO2010/046035, in particular for (2R,3R)-3-{[2-{[4-(S-cyclopropylsulphonimidoyl)phenyl]amino}-5-(trifluoromethyl) pyrimidin-4-yl]oxy}butan-2-ol (Compound A).

Surprisingly, it has now been found that MAD2L2 is suitable as stratification marker for human breast tumour cells in the treatment with the novel pan-CDK inhibitors of WO2010/046035, in particular in the treatment with Compound A, and allows the prediction of sensitivity.

The method according to the invention comprises determination of the MAD2L2 expression as marker for the sensitivity of tumour cells or of tumours for treatment with a CDK inhibitor. To this end, preferably, a quantitative determination is carried out, where the extent of expression of MAD2L2 at the nucleic acid level or/and at the protein level is determined in tumour tissue or in tumour cells and optionally compared to the extent of expression in the surrounding normal tissue.

The extent of expression of MAD2L2 can be determined by standard methods. A preferred embodiment is a determination at the nucleic acid level, e.g. a determination of the amount of transcript. Quantitative determinations of MAD2L2 expression at the nucleic acid level can include, for example, hybridization with labelled MAD2L2-specific probes, nucleic acid amplification reactions, gene chip hybridizations and/or transcript sequencing. Preferred determination methods are quantitative PCR or realtime PCR. Quantitative determinations at the protein level can include immunological detection methods using anti-MAD2L2 antibodies, for example in the Western blot or ELISA format.

The sample in which the MAD2L2 expression is to be determined can originate, for example, from a cell culture or an organism, e.g. a mammal, in particular a human, but also from an experimental animal. Particularly preferably, a determination is carried out on a sample which originates from a culture of tumour cells, in particular of human tumour cells, or from a tumour patient, in particular a human patient or an experimental animal for tumour research. The sample can originate from the tumour itself or from detached tumour cells, e.g. circulating tumour cells from body fluids, e.g. blood.

In a preferred embodiment, the method according to the invention can be applied for selecting a therapy (therapy decision, stratification) in the treatment of a patient, in particular a human patient, during the course of a therapeutic method. Furthermore, the process according to the invention may, in the treatment of an experimental animal, serve to identify and/or characterize novel active compounds. In a further preferred embodiment, the process may be carried out in a cell culture, for example in the context of screening processes.

The method comprises one or more determinations. Preferably, prior to the first administration of the CDK inhibitor, the expression of MAD2L2 is determined in a sample of the cell culture to be examined or the organism to be examined

### Proliferation Assays

### Method 1

This assay was used for the following cell lines: MCF 10A, SK-BR-3, MCF7, HCT 116, HT-29, SW480, Caco-2, MIAPaCa-2, DU145, PC3, HeLa, Caki2, 786-0, A-375, NCI-H460, NCI-H69, NCI-H1975, A549.

Cultivated human tumour cells (originally obtained from ATCC, HeLa-MaTu and HeLa-MaTu-ADR, originally obtained from Epo GmbH, Berlin) were plated in a density of from 1000 to 5000 cells/measurement point, depending on the growth rate of the cell line, in a 96-well multititre plate in 200 μl of growth medium (DMEM/HAMS F12, 2 mM L-glutamine, 10% foetal calf serum). After 24 hours, the cells of one plate (zero-point plate) were stained with crystal violet (see below), while the medium of the other plates was replaced by fresh culture medium (200 μl) with added test substances in various concentrations (0 μM, and in the range of 0.01-30 μM; the final concentration of the solvent dimethyl sulphoxide was 0.5%). The cells were incubated in the presence of the test substances for 4 days. Cell proliferation was determined by staining the cells with crystal violet: the cells were fixated by addition of 20 μl/measuring point of an 11% strength glutaraldehyde solution for 15 min at room temperature. After the fixated cells had been washed three times with water, the plates were dried at room temperature. The cells were stained by addition of 100 μl/measuring point of a 0.1% strength crystal violet solution (pH adjusted to pH 3 by addition of acetic acid). After the stained cells had been washed three times with water, the plates were dried at room temperature. The dye was dissolved by addition of 100 μl/measuring point of a 10% strength acetic acid solution. The absorption was determined photometrically at a wavelength of 595 nm. The change in percent of the cell growth was calculated by normalizing the measured values to the absorption values of the zero-point plate (=0%) and the absorption of the untreated (0 μM) cells (=100%). The measured data were normalized to 0% inhibition (cell proliferation without inhibitor) and 100% inhibition (zero-point plate). The IC₅₀ values were determined using a 4-parameter fit with the aid of proprietary software.

### Method 2

This assay was used for the following cell lines: KPL-1, MDA-MB-453, Hs 578T, MDA-MB-231, MCF 10A, MDA-MB-468, ZR-75-1, T-47D, MDA-MB-435s, DU-4475, BT-20, BT-474, EVSA-T, BT-549, NCI-H460, NCI-H810, NCI-H441, NCI-H1838, NCI-H69, NCI-H2030, NCI-H358, NCI-H1793, NCI-H1048, SK-MES-1, NCI-H2347, NCI-H1975, A549, NCI-H23, NCI-H2170, NCI-H2228, NCI-H661, NCI-H1703, NCI-H1581, NCI-H226, NCI-H1563, NCI-H522, ChaGo-K-1, NCI-H1437. The inhibition of cell proliferation by Compound A was determined using the Vybrant MTT cell proliferation assays from Invitrogen.

### Affymetrix Gene Chip Assay

This assay was used to determine the relative mRNA levels in the tumour cell lines used.

Cultivated human tumour cells were sown at the same cell number/cm² plate area as used in the proliferation assays in 10 cm cell culture plates and incubated in growth medium at 37° C. for 24 hours. The medium was then removed and the cells were washed 2× with in each case 5 ml of phosphate-buffered sodium chloride solution (PBS). The cells were then suspended in 600 μl RLT buffer (Qiagen) with 1% beta-mercaptoethanol. The suspension was homogenised using a QIAShredder in accordance with the manufacturer's instructions. Subsequent RNA extraction was carried out using the RNeasy Mini Kit (Qiagen) in accordance with the manufacturer's instructions. Furthermore, DNase digestion was performed using the RNase-free DNase Kit (Qiagen) in accordance with the manufacturer's instructions.

The final RNA concentration was determined by measuring the optical density at 260 and 280 nm. In addition, the quality of the RNA was checked on an Agilent Bioanalyzer. For further analyses, only RNA having a 28S/18S rRNA ratio of more than 1.0 was used.

5 μg of the RNA samples were used for the synthesis of double-stranded cDNA using the One-Cycle cDNA synthesis kit (Affymetrix) in the presence of a T7-oligo (dT)₂₄ DNA oligonucleotide primer in accordance with the manufacturer's instructions. After the synthesis, the cDNA was purified using the Affymetrix GeneChip Sample Cleanup Module. The purified cDNA was then transcribed using the GeneChip IVT labelling kit (Affymetrix) in the presence of biotinylated ribonucleotides in vitro, giving biotin-labelled cRNA. The labelled cRNA was then purified using the GeneChip Sample Cleanup Module (Affymetrix). The labelled cRNA was determined quantitatively by measuring the optical density at 260 and 280 nm and subjected to a quality check on the Agilent Bioanalyzer.

30 μg of labelled cRNA were fragmented using the fragmentation buffer from the GeneChip Sample Cleanup Module (Affymetrix). 10 μg of fragmented cRNA were then hybridized on a microarray of the human U133 Plus 2.0 type (Affymetrix). The array was then washed and labelled with streptavidin-R-phycoerythrin (SAPE, Molecular Probes). The signal was amplified using a biotinylated anti-streptavidin goat antibody (Vector Laboratories) followed by further labelling with SAPE. The arrays were labelled using the GeneChip Fluidics Station 450 (Affymetrix). The array was then scanned at 570 nm using a confocal laser scanner (GeneChip-3000 Scanner, Affymetrix) and converted into individual quantitative values (1 value for each signal, 40 individual values per gene) using the Affymetrix GeneChip software. The individual values were summarized by implementing the Affymetrix MASS algorithm from Genedata REFINER® to give one value per gene.

The procedure is repeated using in each case three microarrays (replications) for each of the cell lines. The resulting individual values of all genes and replications were normalized to the median of all the values. Subsequently, each value per gene and replication was summarized to one value per gene and cell line by calculating the harmonic mean. Between the mRNA expression values calculated in this manner and the above-described IC-50 values from the proliferation assays, the correlation coefficient according to Pearson between gene and test substance was calculated in each case for all cell lines.

Compound A was examined in the cell lines of Table 1, which serve as examples for the sub-indications listed.

TABLE 1

| Tumour | |
|---|---|
| indication | Cell line |
| Breast | KPL-1, MCF 10A, SK-BR-3, MCF7, MDA-MB-453, Hs |
| carcinoma | 578T, MDA-MB-231, MDA-MB-468, ZR-75-1, T-47D, |
| | MDA-MB-435s, DU-4475, BT-20, BT-474, EVSA-T, BT-549 |
| Colon | HCT 116, HT-29, SW480, Caco-2 |
| carcinoma | |
| Pancreas | MIAPaCa-2 |
| carcinoma | |
| Prostatea | DU145, PC3 |
| carcinom | |
| Cervix | HeLa |
| carcinoma | |
| Renal | Caki2, 786-O |
| carcinoma | |
| Lung | NCI-H460, NCI-H810, NCI-H441, NCI-H1838, NCI-H69, |
| carcinoma | NCI-H2030, NCI-H358, NCI-H1793, NCI-H1048, SK-MES- |
| | 1, NCI-H2347, NCI-H1975, A549, NCI-H23, NCI-H2170, |
| | NCI-H2228, NCI-H661, NCI-H1703, NCI-H1581, NCI- |
| | H226, NCI-H1563, NCI-H522, ChaGo-K-1, NCI-H1437 |
| Melanoma | A-375 |

Table 2 lists 62 genes coding for proteins having a regulatory function in the cell division cycle, which were used for the correlation analysis.

TABLE 2

| Gene symbol | Gene ID | Encoded protein |
|---|---|---|
| CDK1 | 983 | cyclin-dependent kinase 1 |
| CDK2 | 1017 | cyclin-dependent kinase 2 |
| CDK3 | 1018 | cyclin-dependent kinase 3 |
| CDK4 | 1019 | cyclin-dependent kinase 4 |
| CDK6 | 1021 | cyclin-dependent kinase 6 |
| CDK7 | 1022 | cyclin-dependent kinase 7 |
| CDKN1A | 1026 | cyclin-dependent kinase inhibitor 1A |
| | | (p21, Cip1) |
| CDKN1B | 1027 | cyclin-dependent kinase inhibitor 1B |
| | | (p27, Kip1) |
| CDKN1C | 1028 | cyclin-dependent kinase inhibitor 1C |
| | | (p57, Kip2) |
| CDKN2A | 1029 | cyclin-dependent kinase inhibitor 2A (p16) |
| CDKN2B | 1030 | cyclin-dependent kinase inhibitor 2B (p15) |
| CDKN2C | 1031 | cyclin-dependent kinase inhibitor 2C (p18) |
| CDKN2D | 1032 | cyclin-dependent kinase inhibitor 2D (p19) |
| CDKN3 | 1033 | cyclin-dependent kinase inhibitor 3 |
| PLK1 | 5347 | polo-like kinase 1 |
| PLK2 | 10769 | polo-like kinase 2 |
| PLK3 | 1263 | polo-like kinase 3 |
| PLK4 | 10733 | polo-like kinase 4 |
| AURKA | 6790 | aurora kinase A |
| AURKB | 9212 | aurora kinase B |
| CHEK1 | 1111 | CHK1 checkpoint homologue |
| CHEK2 | 11200 | CHK2 checkpoint homologue |
| CCNA1 | 8900 | cyclin A1 |
| CCNA2 | 890 | cyclin A2 |
| CCNB1 | 891 | cyclin B1 |
| CCNB1IP1 | 57820 | cyclin B1 interacting protein 1 |
| CCNB2 | 9133 | cyclin B2 |
| CCNB3 | 85417 | cyclin B3 |
| CCNC | 892 | cyclin C |
| CCND1 | 595 | cyclin D1 |
| CCND2 | 894 | cyclin D2 |
| CCND3 | 896 | cyclin D3 |
| CCNDBP1 | 23582 | cyclin D-type binding-protein 1 |
| CCNE1 | 898 | cyclin E1 |
| CCNE2 | 9134 | cyclin E2 |
| CCNF | 899 | cyclin F |
| CCNG1 | 900 | cyclin G1 |
| CCNG2 | 901 | cyclin G2 |
| CCNH | 902 | cyclin H |
| CCNI | 10983 | cyclin I |
| CCNI2 | 645121 | cyclin I family, member 2 |
| CCNJ | 54619 | cyclin J |
| CCNJL | 79616 | cyclin J-like |
| CCNK | 8812 | cyclin K |
| CCNL1 | 57018 | cyclin L1 |
| CCNL2 | 81669 | cyclin L2 |
| CCNO | 10309 | cyclin O |
| CCNT1 | 904 | cyclin T1 |
| CCNT2 | 905 | cyclin T2 |
| CCNY | 219771 | cyclin Y |
| CCNYL1 | 151195 | cyclin Y-like 1 |
| TTK | 7272 | TTK protein kinase |
| BUB1 | 699 | budding uninhibited by benzimidazoles 1 |
| | | homologue |
| BUB1B | 701 | budding uninhibited by benzimidazoles 1 |
| | | homologue beta |
| BUB3 | 9184 | budding uninhibited by benzimidazoles 3 |
| | | homologue |
| MAD1L1 | 8379 | MAD1 mitotic arrest deficient-like 1 |
| MAD2L1 | 4085 | MAD2 mitotic arrest deficient-like 1 |
| MAD2L1BP | 9587 | MAD2L1 binding protein |
| MAD2L2 | 10459 | MAD2 mitotic arrest deficient-like 2 |
| CDC20 | 991 | cell division cycle 20 homologue |
| CDC20B | 166979 | cell division cycle 20 homologue B |
| WEE1 | 7465 | WEE1 homologue |

Table 3 shows the results of the proliferation assays.

TABLE 3

| | Compound A |
|---|---|
| | IC50 [nM] |
| Breast tumour cell lines | |
| KPL-1 | 6.44 |
| MCF 10A | 20 |
| SK-BR-3 | 13 |
| MCF7 | 15 |
| MDA-MB-453 | 15.1 |
| Hs 578T | 16.8 |
| MDA-MB-231 | 20.2 |
| MDA-MB-468 | 28.8 |
| ZR-75-1 | 32.5 |
| T-47D | 33.8 |
| MDA-MB-435s | 36.4 |
| DU-4475 | 37.3 |
| BT-20 | 38.1 |
| BT-474 | 42.6 |
| EVSA-T | 45 |
| BT-549 | 84.1 |
| Colon carcinoma cell lines | |
| HCT 116 | 18 |
| HT-29 | 29 |
| SW480 | 15 |
| Caco-2 | 16 |
| Pancreas carcinoma cell lines | |
| MIAPaCa-2 | 21 |
| Prostate carcinoma cell lines | |
| DU145 | 8 |
| PC3 | 25 |
| Cervix carcinoma cell lines | |
| HeLa | 12 |
| Renal carcinoma cell lines | |
| Caki2 | 26 |
| 786-O | 20 |
| Melanoma cell lines | |
| A-375 | 14 |
| Lung carcinoma cell lines | |
| NCI-H460 (non-small-cell lung carcinoma) | 27 |
| NCI-H810 (non-small-cell lung carcinoma) | 9.01 |
| NCI-H441 (papillary lung carcinoma) | 10 |
| NCI-H1838 (non-small-cell lung carcinoma) | 15.9 |
| NCI-H69 (small-cell lung carcinoma) | 27 |
| NCI-H2030 (non-small-cell lung carcinoma) | 17.7 |
| NCI-H358 (non-small-cell lung carcinoma) | 19.4 |
| NCI-H1793 (non-small-cell lung carcinoma) | 22.5 |
| NCI-H1048 (small-cell lung carcinoma) | 25 |
| SK-MES-1 (squamous-cell carcinoma) | 26.5 |
| NCI-H2347 (non-small-cell lung carcinoma) | 28 |
| NCI-H1975 (non-small-cell lung carcinoma) | 24 |
| A549 (non-small-cell lung carcinoma) | 31 |
| NCI-H23 (non-small-cell lung carcinoma) | 45.4 |
| NCI-H2170 (small-cell lung carcinoma) | 48.7 |
| NCI-H2228 (non-small-cell lung carcinoma) | 52.1 |
| NCI-H661 (non-small-cell lung carcinoma) | 53.1 |
| NCI-H1703 (non-small-cell lung carcinoma) | 53.6 |
| NCI-H1581 (non-small-cell lung carcinoma) | 53.8 |
| NCI-H226 (mesothelioma) | 54.6 |
| NCI-H1563 (non-small-cell lung carcinoma) | 59.1 |
| NCI-H522 (non-small-cell lung carcinoma) | 65.4 |
| ChaGo-K-1 (undifferentiated bronchial carcinoma) | 69.4 |
| NCI-H1437 (non-small-cell lung carcinoma) | 69.9 |

Table 4 shows the relative mRNA amounts of the 62 cell cycle-regulatory genes in the 51 cell lines examined, determined in Affymetrix gene-chip hybridization studies.

TABLE 4

| | AURKA | AURKB | BUB1B | BUB1 | BUB3 | CCNA1 | CCNA2 | CCNB1IP1 |
|---|---|---|---|---|---|---|---|---|
| KPL1 | 60087 | 19709 | 13265 | 10880 | 78630 | 491 | 18335 | 17308 |
| MCF10A | 34913 | 8307 | 23706 | 14238 | 135711 | 6835 | 28358 | 42206 |
| SK-BR-3 | 127962 | 7888 | 33366 | 18758 | 114446 | 4324 | 41783 | 54014 |
| MCF7 | 66402 | 16299 | 19440 | 13970 | 114589 | 893 | 21914 | 44705 |
| MDAMB453 | 35610 | 6310 | 44008 | 12952 | 163447 | 338 | 32659 | 19811 |
| HS578T | 52223 | 28323 | 50402 | 21942 | 171634 | 189 | 33270 | 39922 |
| MDAMB231 | 132277 | 46600 | 65257 | 63651 | 244291 | 2767 | 77385 | 36196 |
| MDAMB468 | 73003 | 30792 | 53846 | 39361 | 175379 | 112 | 54628 | 86879 |
| ZR751 | 73014 | 15433 | 35067 | 21664 | 137095 | 1018 | 38687 | 37929 |
| T47D | 79613 | 14780 | 53655 | 19656 | 119998 | 280 | 45386 | 15532 |
| MDAMB435S | 99910 | 22673 | 68619 | 37445 | 225787 | 108 | 40529 | 52837 |
| DU4475 | 27257 | 17877 | 31276 | 22899 | 132503 | 361 | 26780 | 11136 |
| BT20 | 59155 | 16032 | 35465 | 19235 | 54212 | 967 | 32077 | 22275 |
| BT474 | 95568 | 8851 | 27409 | 12488 | 61323 | 734 | 18134 | 67693 |
| EVSAT | 132254 | 9140 | 27190 | 11748 | 168616 | 469 | 31774 | 20491 |
| BT549 | 104992 | 30879 | 46425 | 28643 | 146073 | 4155 | 45392 | 40775 |
| HCT116 | 66028 | 36907 | 87813 | 45667 | 273237 | 272 | 49299 | 64683 |
| HT29 | 91034 | 22421 | 55594 | 35853 | 163773 | 463 | 55007 | 45582 |
| SW480 | 72796 | 35221 | 37225 | 36785 | 119547 | 161 | 40022 | 29231 |
| CACO2 | 174500 | 37352 | 52965 | 54378 | 149417 | 276 | 82241 | 78729 |
| MIAPACA2 | 74575 | 33355 | 44046 | 43296 | 148303 | 267 | 34959 | 34158 |
| DU145 | 73547 | 21613 | 55396 | 30225 | 177577 | 3343 | 25582 | 41263 |
| PC3 | 61659 | 12697 | 55837 | 36970 | 159589 | 5657 | 23942 | 27195 |
| HELA | 74457 | 21496 | 73701 | 22733 | 202199 | 563 | 52633 | 29953 |
| Caki2 | 45628 | 5675 | 39081 | 29085 | 90789 | 6407 | 35447 | 15429 |
| 786O | 77549 | 26209 | 49205 | 43467 | 151043 | 3033 | 47113 | 34886 |
| A375 | 43673 | 20524 | 34099 | 15089 | 141130 | 4119 | 26523 | 18131 |
| NCIH226 | 18987 | 4771 | 5859 | 7639 | 37827 | 1040 | 13846 | 16170 |
| NCIH460 | 89625 | 17579 | 32287 | 25536 | 132449 | 247 | 29198 | 56785 |
| NCIH810 | 53140 | 13687 | 32901 | 16399 | 54233 | 637 | 27197 | 44593 |
| NCIH1838 | 32887 | 19843 | 32004 | 14252 | 38809 | 293 | 14462 | 21164 |
| NCIH2030 | 69179 | 25719 | 50663 | 31075 | 76930 | 1439 | 34599 | 13296 |
| NCIH358 | 7896 | 3918 | 9704 | 5131 | 26780 | 545 | 6113 | 31100 |
| NCIH1793 | 31869 | 5416 | 26253 | 10386 | 39172 | 1148 | 18832 | 13474 |
| NCIH2347 | 38717 | 10405 | 18367 | 16974 | 53179 | 730 | 27524 | 22373 |
| NCIH1975 | 24095 | 10956 | 13161 | 8853 | 50520 | 3301 | 13028 | 19691 |
| A549 | 85072 | 36346 | 40617 | 29470 | 145586 | 268 | 37058 | 28256 |
| NCIH23 | 3481 | 1041 | 1904 | 734 | 34821 | 554 | 1983 | 12402 |
| NCIH2228 | 10688 | 1429 | 8638 | 5243 | 35617 | 3023 | 9781 | 17870 |
| NCIH661 | 29728 | 12309 | 17537 | 12839 | 25077 | 722 | 36211 | 21546 |
| NCIH1703 | 41730 | 19655 | 38478 | 18648 | 59750 | 1601 | 36182 | 14451 |
| NCIH1581 | 30583 | 13348 | 19860 | 15472 | 61848 | 1056 | 21693 | 18094 |
| NCIH1563 | 33598 | 2063 | 8042 | 3942 | 34433 | 693 | 11431 | 17888 |
| NCIH522 | 22431 | 6567 | 17392 | 12945 | 39954 | 701 | 18687 | 9338 |
| NCIH1437 | 29997 | 8161 | 29979 | 11391 | 64269 | 1164 | 20668 | 11982 |
| NCIH441 | 35979 | 17896 | 28958 | 15971 | 57455 | 490 | 24281 | 26814 |
| NCIH69 | 14587 | 9410 | 28863 | 8578 | 40234 | 9193 | 20343 | 30090 |
| NCIH1048 | 32538 | 9479 | 39895 | 12935 | 76698 | 1937 | 37710 | 15422 |
| NCIH2170 | 24942 | 7775 | 27835 | 7596 | 30037 | 410 | 31153 | 13755 |
| SKMES1 | 33277 | 7855 | 27593 | 12245 | 46034 | 594 | 32503 | 12895 |
| CHAGOK1 | 55008 | 9057 | 30223 | 10735 | 26993 | 871 | 43567 | 18209 |

| | CCNB1 | CCNB2 | CCNB3 | CCNC | CCND1 | CCND2 | CCNDBP1 |
|---|---|---|---|---|---|---|---|
| KPL1 | 40182 | 33709 | 708 | 20839 | 48200 | 654 | 11047 |
| MCF10A | 89275 | 53637 | 1021 | 125110 | 27295 | 25935 | 41515 |
| SK-BR-3 | 99234 | 62103 | 1177 | 89742 | 25227 | 639 | 23071 |
| MCF7 | 82679 | 41645 | 938 | 85000 | 76589 | 381 | 17308 |
| MDAMB453 | 75501 | 86143 | 1012 | 83630 | 9022 | 556 | 10963 |
| HS578T | 97045 | 75996 | 544 | 69587 | 52776 | 160 | 39611 |
| MDAMB231 | 204466 | 95832 | 1552 | 90089 | 85794 | 689 | 41418 |
| MDAMB468 | 163813 | 103370 | 1046 | 208429 | 19589 | 101 | 22200 |
| ZR751 | 107878 | 77971 | 1666 | 101304 | 62178 | 140 | 11607 |
| T47D | 185519 | 115028 | 1913 | 66247 | 21505 | 464 | 14895 |
| MDAMB435S | 179811 | 159076 | 1262 | 79039 | 69604 | 268 | 28254 |
| DU4475 | 61736 | 52202 | 1126 | 13965 | 5153 | 484 | 22204 |
| BT20 | 75663 | 67015 | 5004 | 59513 | 25778 | 962 | 23759 |
| BT474 | 46558 | 45940 | 1226 | 60702 | 25565 | 463 | 9236 |
| EVSAT | 84263 | 57295 | 1146 | 171163 | 8719 | 161 | 19286 |
| BT549 | 172233 | 41266 | 1046 | 128212 | 9447 | 335 | 2266 |
| HCT116 | 164167 | 94782 | 2284 | 136271 | 71527 | 209 | 30507 |
| HT29 | 166751 | 116312 | 1009 | 117943 | 15940 | 238 | 32229 |
| SW480 | 112696 | 85041 | 1102 | 93170 | 198818 | 13570 | 26414 |
| CACO2 | 243245 | 133942 | 1044 | 194111 | 43688 | 37183 | 29741 |
| MIAPACA2 | 169941 | 95169 | 1203 | 123640 | 19765 | 764 | 20678 |
| DU145 | 111798 | 73889 | 2636 | 86219 | 34482 | 267 | 44471 |
| PC3 | 100313 | 48892 | 722 | 72154 | 68926 | 307 | 23286 |
| HELA | 158606 | 102931 | 855 | 95815 | 10189 | 339 | 22054 |
| Caki2 | 65040 | 42794 | 1479 | 60949 | 24040 | 432 | 14974 |
| 786O | 103593 | 82152 | 615 | 105183 | 68088 | 357 | 28043 |
| A375 | 90572 | 50907 | 1203 | 46133 | 60715 | 734 | 18184 |
| NCIH226 | 26358 | 32041 | 1006 | 42815 | 30528 | 4643 | 11841 |
| NCIH460 | 108118 | 52566 | 1986 | 68165 | 10759 | 177 | 18456 |
| NCIH810 | 49505 | 52117 | 676 | 44269 | 2435 | 623 | 28322 |
| NCIH1838 | 38211 | 37795 | 1988 | 31341 | 44366 | 656 | 23656 |
| NCIH2030 | 64336 | 44768 | 2225 | 30516 | 15321 | 668 | 24893 |
| NCIH358 | 12239 | 15280 | 2986 | 19233 | 35286 | 319 | 11794 |
| NCIH1793 | 38759 | 25575 | 5233 | 23796 | 16101 | 703 | 11431 |
| NCIH2347 | 81585 | 32726 | 1850 | 38105 | 76835 | 2645 | 21251 |
| NCIH1975 | 36185 | 17669 | 1812 | 30408 | 22628 | 919 | 18635 |
| A549 | 157943 | 98461 | 1233 | 78118 | 43552 | 278 | 20600 |
| NCIH23 | 2278 | 2392 | 940 | 22242 | 22776 | 1077 | 26398 |
| NCIH2228 | 24662 | 10757 | 1110 | 24504 | 24771 | 4302 | 20331 |
| NCIH661 | 23425 | 24205 | 4548 | 24684 | 3656 | 883 | 12882 |
| NCIH1703 | 79569 | 41934 | 8474 | 49850 | 6373 | 92426 | 15653 |
| NCIH1581 | 34744 | 21939 | 3052 | 38088 | 22083 | 59479 | 6609 |
| NCIH1563 | 18305 | 10696 | 2026 | 39449 | 17891 | 875 | 16138 |
| NCIH522 | 24099 | 15579 | 2037 | 20182 | 9779 | 606 | 5687 |
| NCIH1437 | 47843 | 30182 | 1162 | 26696 | 36427 | 735 | 8907 |
| NCIH441 | 50870 | 33166 | 952 | 32001 | 37893 | 702 | 23983 |
| NCIH69 | 51248 | 40047 | 1320 | 21218 | 975 | 501 | 5518 |
| NCIH1048 | 53259 | 36826 | 3074 | 37329 | 8030 | 753 | 20986 |
| NCIH2170 | 31099 | 20680 | 1318 | 46194 | 22041 | 582 | 9402 |
| SKMES1 | 39311 | 28313 | 1564 | 45079 | 48918 | 858 | 20602 |
| CHAGOK1 | 42782 | 32033 | 1481 | 34545 | 10716 | 1972 | 11709 |

| | CCNE1 | CCNE2 | CCNF | CCNG1 | CCNG2 | CCNH | CCN1 | CCNJL | CCNJ |
|---|---|---|---|---|---|---|---|---|---|
| KPL1 | 7449 | 10202 | 8871 | 51799 | 10803 | 21255 | 62025 | 495 | 2582 |
| MCF10A | 5870 | 8917 | 6513 | 214311 | 46371 | 36521 | 116413 | 881 | 3918 |
| SK-BR-3 | 11395 | 9917 | 11550 | 40298 | 25045 | 25747 | 93707 | 552 | 1630 |
| MCF7 | 7982 | 27985 | 9084 | 114479 | 37286 | 38805 | 120523 | 1421 | 6153 |
| MDAMB453 | 3902 | 35004 | 12953 | 76736 | 63267 | 25735 | 109657 | 275 | 3102 |
| HS578T | 9497 | 30371 | 7353 | 122062 | 42063 | 44285 | 112047 | 276 | 3067 |
| MDAMB231 | 12610 | 46334 | 10656 | 71751 | 24820 | 57429 | 102215 | 4203 | 6713 |
| MDAMB468 | 14018 | 32314 | 16590 | 200683 | 16788 | 45188 | 110454 | 2233 | 4752 |
| ZR751 | 7913 | 33222 | 10848 | 120001 | 48045 | 37453 | 126774 | 773 | 4561 |
| T47D | 5209 | 51290 | 10496 | 106730 | 23705 | 53287 | 66332 | 1562 | 6148 |
| MDAMB435S | 9911 | 26574 | 9343 | 76112 | 7885 | 69644 | 45102 | 1937 | 2488 |
| DU4475 | 12821 | 22050 | 10181 | 67147 | 5496 | 8263 | 16196 | 722 | 3048 |
| BT20 | 8064 | 20885 | 7976 | 20197 | 8817 | 20775 | 66365 | 241 | 1525 |
| BT474 | 3386 | 37458 | 8096 | 42635 | 91312 | 8382 | 160602 | 2035 | 6084 |
| EVSAT | 8746 | 89382 | 9298 | 80607 | 9277 | 19844 | 81986 | 529 | 2553 |
| BT549 | 17171 | 111778 | 7348 | 69766 | 12548 | 57541 | 78890 | 667 | 8601 |
| HCT116 | 25789 | 31245 | 17540 | 191267 | 13514 | 63028 | 96242 | 1472 | 9666 |
| HT29 | 12079 | 16979 | 11710 | 172609 | 17408 | 59099 | 104601 | 1864 | 6640 |
| SW480 | 5985 | 19425 | 8542 | 98231 | 13603 | 38635 | 69164 | 2336 | 5009 |
| CACO2 | 20296 | 19464 | 15387 | 244602 | 9948 | 62280 | 82075 | 3075 | 12189 |
| MIAPACA2 | 16987 | 12615 | 10311 | 168313 | 7779 | 73449 | 86675 | 689 | 5850 |
| DU145 | 7817 | 20946 | 5910 | 77921 | 7782 | 58070 | 85762 | 1967 | 4747 |
| PC3 | 4748 | 26708 | 4891 | 78028 | 13221 | 87461 | 54438 | 2358 | 4231 |
| HELA | 10308 | 23960 | 11588 | 110737 | 12017 | 66020 | 71595 | 1688 | 3268 |
| Caki2 | 8784 | 11005 | 5882 | 96877 | 17575 | 15795 | 65311 | 1572 | 2346 |
| 786O | 6578 | 27305 | 10993 | 106597 | 14078 | 37659 | 117412 | 1138 | 5194 |
| A375 | 6802 | 16588 | 7715 | 72688 | 4234 | 45503 | 42682 | 2150 | 5311 |
| NCIH226 | 2092 | 3712 | 4330 | 49097 | 4425 | 21703 | 52648 | 203 | 1367 |
| NCIH460 | 7347 | 14398 | 5507 | 157712 | 4716 | 54116 | 63949 | 1931 | 5807 |
| NCIH810 | 79712 | 10930 | 20114 | 22510 | 12404 | 26656 | 86840 | 1344 | 11165 |
| NCIH1838 | 4239 | 8895 | 4221 | 23900 | 12462 | 14388 | 56493 | 1521 | 3432 |
| NCIH2030 | 10546 | 8709 | 6722 | 23169 | 20622 | 34637 | 72705 | 984 | 2391 |
| NCIH358 | 1256 | 2504 | 1835 | 13800 | 25035 | 16565 | 69362 | 1578 | 1863 |
| NCIH1793 | 6466 | 10129 | 3011 | 32761 | 10503 | 27326 | 59685 | 2999 | 2764 |
| NCIH2347 | 8457 | 14631 | 5112 | 41312 | 48651 | 26550 | 87779 | 2999 | 3308 |
| NCIH1975 | 4049 | 4237 | 3443 | 30031 | 16911 | 32740 | 55462 | 1033 | 3053 |
| A549 | 17739 | 10115 | 9795 | 102327 | 4658 | 45722 | 53411 | 2739 | 6158 |
| NCIH23 | 3118 | 772 | 1396 | 51588 | 6894 | 20820 | 84235 | 2319 | 4805 |
| NCIH2228 | 7369 | 5549 | 2463 | 29830 | 6497 | 42934 | 78040 | 6555 | 4769 |
| NCIH661 | 7143 | 1815 | 5024 | 21321 | 7234 | 5650 | 51006 | 1229 | 2787 |
| NCIH1703 | 6553 | 16143 | 6937 | 60337 | 9326 | 27072 | 71597 | 1105 | 5361 |
| NCIH1581 | 8770 | 7106 | 6765 | 20828 | 3270 | 11822 | 56179 | 557 | 7318 |
| NCIH1563 | 9361 | 9513 | 3375 | 45050 | 25317 | 23017 | 62919 | 2298 | 2366 |
| NCIH522 | 23722 | 12804 | 4506 | 14699 | 3388 | 10975 | 50119 | 1121 | 5797 |
| NCIH1437 | 6992 | 16572 | 6736 | 16872 | 2742 | 19023 | 62323 | 3196 | 3954 |
| NCIH441 | 5605 | 15168 | 7040 | 38753 | 47990 | 17369 | 80164 | 3972 | 2637 |
| NCIH69 | 7943 | 31851 | 9302 | 25182 | 6495 | 43103 | 52174 | 1166 | 4430 |
| NCIH1048 | 16545 | 23224 | 5268 | 37928 | 26799 | 25356 | 75506 | 1868 | 5855 |
| NCIH2170 | 9603 | 9859 | 3955 | 29639 | 9605 | 18626 | 85821 | 1173 | 4716 |
| SKMES1 | 3782 | 7070 | 5691 | 22203 | 16785 | 24370 | 73256 | 3022 | 2729 |
| CHAGOK1 | 19286 | 6716 | 6549 | 27293 | 11383 | 12891 | 62833 | 722 | 117 |

| | CCNK | CCNL1 | CCNL2 | CCNO | CCNT1 | CCNT2 |
|---|---|---|---|---|---|---|
| KPL1 | 28501 | 14840 | 23740 | 4545 | 3389 | 3559 |
| MCF10A | 19601 | 19360 | 43684 | 3926 | 3250 | 9435 |
| SK-BR-3 | 11365 | 10480 | 21899 | 1443 | 2279 | 5827 |
| MCF7 | 41185 | 19297 | 22037 | 2471 | 3104 | 9067 |
| MDAMB453 | 23207 | 13118 | 18166 | 13179 | 814 | 9630 |
| HS578T | 18921 | 23818 | 37708 | 2371 | 2046 | 6298 |
| MDAMB231 | 24617 | 12932 | 23517 | 4387 | 2164 | 6686 |
| MDAMB468 | 27090 | 19304 | 16337 | 2769 | 1810 | 7644 |
| ZR751 | 30143 | 20407 | 22858 | 2196 | 2661 | 15250 |
| T47D | 30258 | 15232 | 18540 | 9622 | 3401 | 7187 |
| MDAMB435S | 17254 | 26770 | 11611 | 9103 | 2255 | 5126 |
| DU4475 | 11491 | 14112 | 12759 | 4848 | 2824 | 6297 |
| BT20 | 21944 | 10685 | 11214 | 3110 | 2559 | 5657 |
| BT474 | 19793 | 14258 | 15422 | 1272 | 2314 | 9358 |
| EVSAT | 27309 | 10448 | 25481 | 4584 | 3189 | 8913 |
| BT549 | 24518 | 13633 | 23912 | 945 | 1247 | 4265 |
| HCT116 | 33717 | 24582 | 28895 | 7116 | 3789 | 9434 |
| HT29 | 21458 | 14890 | 24108 | 4225 | 2654 | 10445 |
| SW480 | 27180 | 27006 | 29740 | 4290 | 1871 | 7659 |
| CACO2 | 33688 | 23952 | 25959 | 3639 | 4006 | 15584 |
| MIAPACA2 | 26089 | 25892 | 35065 | 5496 | 2643 | 8573 |
| DU145 | 28433 | 22827 | 24339 | 3667 | 2729 | 6938 |
| PC3 | 21539 | 17765 | 39738 | 3323 | 2904 | 7894 |
| HELA | 18645 | 11450 | 29199 | 4219 | 2729 | 7807 |
| Caki2 | 15165 | 10863 | 14418 | 2892 | 1270 | 4203 |
| 786O | 22106 | 13183 | 28445 | 2692 | 3506 | 8151 |
| A375 | 14694 | 13970 | 18289 | 5070 | 1861 | 2947 |
| NCIH226 | 12448 | 12322 | 15985 | 3093 | 2494 | 4019 |
| NCIH460 | 21874 | 11758 | 16817 | 4538 | 3205 | 10418 |
| NCIH810 | 21196 | 16688 | 14667 | 1030 | 2892 | 6702 |
| NCIH1838 | 17065 | 10212 | 12636 | 5654 | 1175 | 1843 |
| NCIH2030 | 22097 | 11908 | 14229 | 1068 | 2045 | 5731 |
| NCIH358 | 10432 | 8346 | 19168 | 2115 | 1161 | 2958 |
| NCIH1793 | 15195 | 10387 | 5361 | 4420 | 1668 | 2284 |
| NCIH2347 | 21535 | 11444 | 11349 | 4468 | 2575 | 3745 |
| NCIH1975 | 20000 | 6624 | 9230 | 1288 | 1199 | 4793 |
| A549 | 27901 | 15024 | 15727 | 3351 | 2510 | 6088 |
| NCIH23 | 11906 | 17412 | 26304 | 7122 | 1050 | 6971 |
| NCIH2228 | 26117 | 11200 | 17110 | 4965 | 2814 | 5968 |
| NCIH661 | 11568 | 5807 | 12103 | 3402 | 1571 | 4718 |
| NCIH1703 | 20939 | 9632 | 21355 | 4100 | 1253 | 5248 |
| NCIH1581 | 30563 | 7059 | 17103 | 1121 | 1978 | 5716 |
| NCIH1563 | 13930 | 8030 | 6496 | 1535 | 526 | 6381 |
| NCIH522 | 27379 | 7684 | 11692 | 3590 | 1162 | 4534 |
| NCIH1437 | 24478 | 10165 | 8057 | 4820 | 1669 | 4352 |
| NCIH441 | 19322 | 10797 | 27111 | 2112 | 1581 | 5581 |
| NCIH69 | 10509 | 5265 | 7996 | 15777 | 910 | 2368 |
| NCIH1048 | 16966 | 7060 | 12469 | 3591 | 1626 | 5823 |
| NCIH2170 | 16668 | 9156 | 7773 | 6297 | 1177 | 6888 |
| SKMES1 | 21037 | 10514 | 15374 | 4872 | 918 | 3838 |
| CHAGOK1 | 16927 | 8711 | 23642 | 5816 | 18796 | 4704 |

| | CCNYL1 | CCNY | CDC20B | CDC20 | CDK1 | CDK2 | CDK3 | CDK4 | CDK6 |
|---|---|---|---|---|---|---|---|---|---|
| KPL1 | 4381 | 8952 | 618 | 33235 | 40424 | 30789 | 2204 | 63959 | 11946 |
| MCF10A | 22824 | 26926 | 245 | 35859 | 59001 | 16264 | 3729 | 66301 | 42299 |
| SK-BR-3 | 4516 | 25848 | 300 | 54435 | [symbol] 44149 | 22037 | 5111 | 72053 | 6951 |
| MCF7 | 8307 | 21784 | 564 | 22827 | 53922 | 34536 | 4038 | 118657 | 3580 |
| MDAMB453 | 9161 | 12474 | 642 | 34004 | 86036 | 24896 | 2719 | 81076 | 643 |
| HS578T | 33991 | 38961 | 353 | 79236 | [symbol] 13081 | 27550 | 2579 | 136997 | 66662 |
| MDAMB231 | 20364 | 34496 | 429 | 89770 | [symbol] 43209 | 43589 | 3563 | 96622 | 40513 |
| MDAMB468 | 32925 | 15331 | 125 | 183758 | 246851 | 17121 | 6169 | 64686 | 17174 |
| ZR751 | 8331 | 29683 | 317 | 27388 | 134235 | 23529 | 6044 | 140821 | 189 |
| T47D | 5114 | 26514 | 3072 | 45820 | 202034 | 41399 | 5669 | 92379 | 1395 |
| MDAMB435S | 17920 | 25082 | 516 | 75099 | 117373 | 25968 | 3979 | 133857 | 22662 |
| DU4475 | 4892 | 13443 | 1005 | 47589 | 84856 | 23132 | 1218 | 53383 | 49060 |
| BT20 | 14106 | 18946 | 1012 | 40625 | 72243 | 30606 | 4721 | 46717 | 4992 |
| BT474 | 2214 | 14111 | 471 | 31884 | 53753 | 27230 | 3984 | 74764 | 2401 |
| EVSAT | 12778 | 27286 | 443 | 29702 | 92306 | 36609 | 4526 | 125415 | 85137 |
| BT549 | 5762 | 34120 | 423 | 84470 | 22109 | 47654 | 2219 | 152880 | 27694 |
| HCT116 | 11126 | 36816 | 236 | [symbol] 20371 | [symbol] 08297 | 72131 | 3398 | 168971 | 37281 |
| HT29 | 19596 | 21710 | 424 | 58384 | [symbol] 63524 | 39319 | 4528 | 130277 | 42756 |
| SW480 | 17271 | 19031 | 402 | 59401 | [symbol] 1434 | 40373 | 3938 | 108253 | 33414 |
| CACO2 | 54684 | 38499 | 496 | 62658 | [symbol] 12205 | 51182 | 4403 | 167659 | 61924 |
| MIAPACA2 | 9339 | 25666 | 484 | 96197 | [symbol] 5663 | 25787 | 4845 | 99734 | 14593 |
| DU145 | 29595 | 22511 | 460 | 54735 | [symbol] 14283 | 45604 | 4465 | 70300 | 5042 |
| PC3 | 20138 | 56121 | 260 | 48606 | [symbol] 70006 | 27756 | 5063 | 95333 | 22119 |
| HELA | 13064 | 15770 | 467 | 67694 | [symbol] 32487 | 54138 | 2914 | 102373 | 52799 |
| Caki2 | 39002 | 11400 | 726 | 34668 | [symbol] 2476 | 28952 | 3832 | 90877 | 32888 |
| 786O | 28505 | 32078 | 705 | 93204 | [symbol] 75500 | 30449 | 4491 | 131095 | 113604 |
| A375 | 6063 | 15591 | 230 | 68098 | [symbol] 5382 | 38886 | 3910 | 106783 | 52547 |
| NCIH226 | 33613 | 9383 | 1052 | 16134 | [symbol] 2521 | 25029 | 3017 | 63673 | 40535 |
| NCIH460 | 46119 | 10738 | 377 | 31250 | [symbol] 18959 | 20679 | 3457 | 99639 | 31260 |
| NCIH810 | 3909 | 19495 | 512 | 53693 | [symbol] 5896 | 28197 | 4596 | 78474 | 14729 |
| NCIH1838 | 12699 | 11087 | 916 | 22319 | [symbol] 3503 | 16031 | 3673 | 78950 | 8906 |
| NCIH2030 | 31179 | 8922 | 803 | 56552 | [symbol] 3601 | 43962 | 4754 | 52759 | 22531 |
| NCIH358 | 5535 | 7668 | 510 | 7952 | [symbol] 090 | 12091 | 2225 | 43895 | 7823 |
| NCIH1793 | 4702 | 5693 | 1072 | 18225 | [symbol] 8882 | 23275 | 4238 | 58423 | 11879 |
| NCIH2347 | 8619 | 11917 | 728 | 35534 | [symbol] 2441 | 25349 | 5335 | 60657 | 9168 |
| NCIH1975 | 11612 | 7709 | 735 | 18974 | [symbol] 7172 | 16886 | 5056 | 59398 | 3716 |
| A549 | 23016 | 21726 | 543 | 55076 | [symbol] 35093 | 30920 | 4949 | 112191 | 35280 |
| NCIH23 | 4340 | 19972 | 820 | 1252 | [symbol] 749 | 9152 | 6186 | 54922 | 15207 |
| NCIH2228 | 11660 | 11961 | 933 | 5327 | [symbol] 342 | 11627 | 5361 | 62607 | 955 |
| NCIH661 | 30038 | 12032 | 1031 | 21485 | [symbol] 6109 | 20143 | 4672 | 53652 | 15138 |
| NCIH1703 | 47804 | 14061 | 769 | 52930 | [symbol] 7433 | 46288 | 7827 | 65652 | 12605 |
| NCIH1581 | 10675 | 15903 | 777 | 29826 | [symbol] 9103 | 31977 | 8731 | 81174 | 18862 |
| NCIH1563 | 5829 | 11675 | 1111 | 11126 | [symbol] 5374 | 11776 | 4762 | 30387 | 11549 |
| NCIH522 | 8089 | 7826 | 959 | 31058 | [symbol] 9561 | 29262 | 4532 | 54728 | 293 |
| NCIH1437 | 12776 | 13836 | 978 | 18398 | [symbol] 0832 | 32876 | 7254 | 53515 | 5346 |
| NCIH441 | 18398 | 17188 | 518 | 31378 | [symbol] 4901 | 23962 | 4971 | 49473 | 9511 |
| NCIH69 | 2065 | 8741 | 2025 | 9531 | [symbol] 4613 | 29635 | 4387 | 62350 | 8083 |
| NCIH1048 | 3243 | 9072 | 604 | 36219 | [symbol] 3358 | 40307 | 5162 | 44135 | 6357 |
| NCIH2170 | 10278 | 8593 | 907 | 16078 | [symbol] 9879 | 12108 | 2316 | 33518 | 14734 |
| SKMES1 | 11376 | 11238 | 711 | 29731 | [symbol] 4094 | 17433 | 5260 | 41812 | 11924 |
| CHAGOK1 | 7092 | 18339 | 591 | 33236 | 3856 | 21646 | 6313 | 31918 | 17577 |

| | CDK7 | CDKN1A | CDKN1B | CDKN1C | CDKN2A | CDKN2B |
|---|---|---|---|---|---|---|
| KPL1 | 16233 | 17886 | 22503 | 627 | 2195 | 1521 |
| MCF10A | 38197 | 48775 | 29848 | 12411 | 941 | 49 |
| SK-BR-3 | 31424 | 3404 | 42190 | 463 | 15080 | 5868 |
| MCF7 | 33265 | 73843 | 74835 | 1137 | 970 | 3597 |
| MDAMB453 | 17398 | 1407 | 81115 | 2940 | 7086 | 211 |
| HS578T | 28058 | 19024 | 46542 | 1533 | 1324 | 16642 |
| MDAMB231 | 53269 | 26918 | 47734 | 9120 | 577 | 131 |
| MDAMB468 | 31187 | 7811 | 29242 | 2736 | 83704 | 8877 |
| ZR751 | 39275 | 40616 | 87423 | 485 | 3393 | 1155 |
| T47D | 39341 | 11414 | 29061 | 1361 | 28830 | 3927 |
| MDAMB435S | 39980 | 6012 | 16144 | 4805 | 77563 | 2651 |
| DU4475 | 19713 | 12833 | 9337 | 40158 | 12537 | 505 |
| BT20 | 10775 | 4357 | 42073 | 2480 | 1476 | 77 |
| BT474 | 15440 | 3156 | 60831 | 2520 | 5253 | 304 |
| EVSAT | 14140 | 3073 | 56488 | 1183 | 10447 | 1283 |
| BT549 | 35473 | 8706 | 40908 | 1179 | 281653 | 6614 |
| HCT116 | 59164 | 54992 | 44485 | 9732 | 35122 | 2066 |
| HT29 | 43029 | 9195 | 39008 | 2614 | 18519 | 1281 |
| SW480 | 30312 | 21191 | 47872 | 12401 | 27809 | 3967 |
| CACO2 | 64311 | 4098 | 122817 | 8105 | 19216 | 1397 |
| MIAPACA2 | 43369 | 5089 | 56844 | 2133 | 956 | 118 |
| DU145 | 33965 | 16718 | 52334 | 3918 | 212852 | 7490 |
| PC3 | 16253 | 9207 | 22520 | 5096 | 65026 | 11397 |
| HELA | 26141 | 16508 | 70677 | 21876 | 269053 | 18919 |
| Caki2 | 12960 | 34120 | 31731 | 2094 | 2422 | 48 |
| 786O | 15712 | 40389 | 50535 | 2067 | 978 | 49 |
| A375 | 26831 | 18389 | 20029 | 3202 | 3200 | 69 |
| NCIH226 | 14649 | 46497 | 16403 | 7021 | 3020 | 5733 |
| NCIH460 | 40611 | 16965 | 19523 | 520 | 940 | 82 |
| NCIH810 | 10384 | 1064 | 39299 | 17439 | 42443 | 651 |
| NCIH1838 | 8703 | 9049 | 24938 | 11398 | 1232 | 22 |
| NCIH2030 | 15302 | 14066 | 21265 | 3391 | 20932 | 1865 |
| NCIH358 | 9948 | 18077 | 14809 | 4313 | 21755 | 8232 |
| NCIH1793 | 12608 | 7921 | 14837 | 1707 | 1549 | 21194 |
| NCIH2347 | 32867 | 22664 | 27749 | 10207 | 25842 | 7015 |
| NCIH1975 | 18739 | 6270 | 22548 | 13419 | 9295 | 3985 |
| A549 | 34367 | 31946 | 32216 | 1112 | 1146 | 95 |
| NCIH23 | 12427 | 1089 | 21061 | 5559 | 24470 | 9133 |
| NCIH2228 | 16784 | 15211 | 30886 | 17106 | 1452 | 32 |
| NCIH661 | 7877 | 4228 | 13023 | 5662 | 17914 | 1549 |
| NCIH1703 | 20916 | 3841 | 40597 | 2583 | 44867 | 2044 |
| NCIH1581 | 7804 | 3113 | 21542 | 6209 | 20216 | 1005 |
| NCIH1563 | 9263 | 37245 | 22152 | 6741 | 1830 | 49 |
| NCIH522 | 7803 | 11968 | 5846 | 1975 | 80732 | 4494 |
| NCIH1437 | 12071 | 4730 | 24149 | 1538 | 1215 | 14 |
| NCIH441 | 13934 | 10841 | 34381 | 3838 | 10589 | 2317 |
| NCIH69 | 7868 | 45686 | 23232 | 3708 | 44704 | 1288 |
| NCIH1048 | 9158 | 14920 | 33676 | 18523 | 61651 | 8772 |
| NCIH2170 | 7986 | 3099 | 37263 | 512 | 2025 | 29 |
| SKMES1 | 11796 | 12121 | 19711 | 8291 | 1067 | 52 |
| CHAGOK1 | 18319 | 2154 | 27678 | 5405 | 12925 | 1386 |

| | CDKN2C | CDKN2D | CDKN3 | CHEK1 | CHEK2 | MAD1L1 | MAD2L1BP | MAD2L1 | MAD2L2 |
|---|---|---|---|---|---|---|---|---|---|
| KPL1 | 5301 | 592 | 33679 | 4449 | 1632 | 4841 | 8948 | 21176 | 14196 |
| MCF10A | 7953 | 886 | 30518 | 17467 | 7251 | 3052 | 17355 | 56760 | 12673 |
| SK-BR-3 | 50475 | 1758 | 72086 | 14959 | 15066 | 5870 | 14552 | 84768 | 15033 |
| MCF7 | 10087 | 1026 | 48465 | 11110 | 8704 | 5092 | 20952 | 69586 | 16809 |
| MDAMB453 | 21494 | 3350 | 71765 | 20120 | 13517 | 3215 | 16317 | 90782 | 17475 |
| HS578T | 95283 | 5973 | 59812 | 24882 | 8391 | 28612 | 28412 | 63817 | 44555 |
| MDAMB231 | 53073 | 14064 | 128476 | 33045 | 6317 | 12959 | 29722 | 134623 | 28981 |
| MDAMB468 | 44520 | 16944 | 97918 | 48012 | 12291 | 14557 | 24655 | 64734 | 17338 |
| ZR751 | 18986 | 2400 | 55498 | 10010 | 15010 | 4841 | 26278 | 85402 | 18598 |
| T47D | 36520 | 2050 | 118007 | 39527 | 12071 | 4150 | 16084 | 69423 | 20773 |
| MDAMB435S | 27417 | 4978 | 84390 | 32073 | 13132 | 46533 | 43056 | 106676 | 38135 |
| DU4475 | 4305 | 7774 | 39818 | 11659 | 2880 | 30419 | 7945 | 44753 | 47627 |
| BT20 | 24453 | 1003 | 53910 | 12882 | 5640 | 4834 | 13954 | 59566 | 17156 |
| BT474 | 10043 | 1151 | 44173 | 13646 | 6666 | 4360 | 15007 | 43592 | 21612 |
| EVSAT | 23948 | 1495 | 68301 | 25554 | 26731 | 3212 | 27993 | 90825 | 48301 |
| BT549 | 194081 | 2428 | 69592 | 23070 | 15953 | 6370 | 19238 | 113278 | 74131 |
| HCT116 | 6043 | 2432 | 61149 | 30911 | 17912 | 6212 | 26003 | 142163 | 42807 |
| HT29 | 10348 | 1468 | 61685 | 30958 | 16409 | 4686 | 17715 | 142199 | 20390 |
| SW480 | 10928 | 1460 | 89492 | 27923 | 5250 | 11118 | 12309 | 86735 | 34576 |
| CACO2 | 2789 | 1039 | 79792 | 56214 | 15305 | 5458 | 26333 | 126843 | 25147 |
| MIAPACA2 | 26051 | 2026 | 90363 | 17241 | 19901 | 15437 | 20673 | 84369 | 48139 |
| DU145 | 37692 | 1264 | 74630 | 23958 | 16146 | 3283 | 29583 | 59734 | 23923 |
| PC3 | 44158 | 3135 | 138850 | 21906 | 8125 | 4682 | 16238 | 82311 | 23028 |
| HELA | 121220 | 1472 | 58582 | 13764 | 12594 | 5549 | 13497 | 98598 | 27530 |
| Caki2 | 38288 | 456 | 55322 | 5236 | 3638 | 1368 | 10154 | 82337 | 12493 |
| 786O | 61859 | 2019 | 60653 | 21260 | 9191 | 4275 | 17497 | 126796 | 35884 |
| A375 | 23971 | 1896 | 21361 | 15167 | 5261 | 4876 | 9670 | 54050 | 14856 |
| NCIH226 | 12000 | 457 | 12715 | 4480 | 1936 | 3631 | 6463 | 29207 | 10843 |
| NCIH460 | 15460 | 861 | 52204 | 13749 | 16543 | 3770 | 12572 | 70461 | 18076 |
| NCIH810 | 26361 | 4148 | 28540 | 9799 | 6610 | 13164 | 13379 | 82085 | 17351 |
| NCIH1838 | 19457 | 2385 | 37599 | 11013 | 6007 | 10721 | 18382 | 38632 | 14779 |
| NCIH2030 | 31745 | 1316 | 77476 | 17810 | 11256 | 4315 | 16730 | 63120 | 20314 |
| NCIH358 | 2978 | 811 | 10373 | 4377 | 2687 | 2302 | 7778 | 28761 | 5790 |
| NCIH1793 | 29263 | 757 | 37528 | 4437 | 4627 | 2706 | 3998 | 39807 | 6678 |
| NCIH2347 | 9414 | 2273 | 46740 | 11895 | 2861 | 3715 | 9649 | 51794 | 10778 |
| NCIH1975 | 11153 | 860 | 22119 | 9259 | 3351 | 4156 | 6866 | 28054 | 9920 |
| A549 | 15550 | 754 | 72577 | 18539 | 7978 | 3631 | 12320 | 87282 | 17876 |
| NCIH23 | 591 | 314 | 1730 | 2410 | 2863 | 2910 | 8499 | 6803 | 5496 |
| NCIH2228 | 3683 | 454 | 20158 | 7261 | 2784 | 4287 | 10381 | 14942 | 12681 |
| NCIH661 | 14055 | 349 | 25401 | 7646 | 3631 | 6124 | 7484 | 51939 | 22198 |
| NCIH1703 | 22165 | 1306 | 51213 | 14004 | 10886 | 6446 | 16155 | 58119 | 32471 |
| NCIH1581 | 27076 | 281 | 35495 | 14836 | 5182 | 3738 | 10878 | 57567 | 30903 |
| NCIH1563 | 23670 | 731 | 19393 | 4992 | 3926 | 2293 | 7637 | 21475 | 4905 |
| NCIH522 | 25713 | 770 | 14011 | 15357 | 4046 | 2734 | 16046 | 47912 | 26801 |
| NCIH1437 | 7590 | 1149 | 48497 | 19166 | 4238 | 2805 | 7674 | 52435 | 10532 |
| NCIH441 | 16038 | 1345 | 54481 | 11813 | 5527 | 3062 | 11044 | 54583 | 13096 |
| NCIH69 | 62350 | 4890 | 30152 | 11463 | 5383 | 1824 | 9499 | 57809 | 19871 |
| NCIH1048 | 33156 | 3199 | 45362 | 6950 | 3987 | 2675 | 11361 | 59474 | 20308 |
| NCIH2170 | 4690 | 572 | 31098 | 10264 | 5525 | 1885 | 8737 | 79720 | 6074 |
| SKMES1 | 16719 | 1311 | 30074 | 11612 | 4508 | 7353 | 8146 | 54256 | 17518 |
| CHAGOK1 | 13277 | 1276 | 32460 | 11366 | 7571 | 2689 | 6736 | 63418 | 19372 |

| | PLK1 | PLK2 | PLK3 | PLK4 | TTK | WEE1 |
|---|---|---|---|---|---|---|
| KPL1 | 13342 | 70636 | 2389 | 7400 | 8079 | 9022 |
| MCF10A | 10151 | 40414 | 3521 | 8129 | 18357 | 25568 |
| SK-BR-3 | 14681 | 5745 | 1434 | 11059 | 27120 | 53706 |
| MCF7 | 11125 | 35947 | 1073 | 10308 | 16215 | 20028 |
| MDAMB453 | 16154 | 51545 | 967 | 15148 | 18763 | 18784 |
| HS578T | 16739 | 57018 | 2159 | 14428 | 33352 | 32375 |
| MDAMB231 | 23060 | 133894 | 1810 | 33258 | 46790 | 48909 |
| MDAMB468 | 41705 | 77176 | 1365 | 17402 | 72655 | 39782 |
| ZR751 | 22831 | 13121 | 853 | 14573 | 22615 | 28583 |
| T47D | 15848 | 79611 | 354 | 11907 | 31549 | 29847 |
| MDAMB435S | 32740 | 20664 | 709 | 20888 | 71378 | 37345 |
| DU4475 | 16735 | 7491 | 1915 | 10075 | 14073 | 13893 |
| BT20 | 9937 | 28290 | 758 | 10014 | 19635 | 40096 |
| BT474 | 7568 | 17469 | 510 | 9343 | 19375 | 40387 |
| EVSAT | 7636 | 3201 | 718 | 16233 | 31021 | 50256 |
| BT549 | 21315 | 136091 | 1637 | 17477 | 40632 | 17143 |
| HCT116 | 28728 | 33560 | 5623 | 25083 | 49463 | 68026 |
| HT29 | 24951 | 70335 | 2365 | 23088 | 28163 | 40607 |
| SW480 | 16381 | 17143 | 2054 | 16663 | 38854 | 30307 |
| CACO2 | 27243 | 6094 | 788 | 19952 | 48037 | 66877 |
| MIAPACA2 | 21334 | 220992 | 2593 | 10082 | 27472 | 22020 |
| DU145 | 13762 | 34232 | 1090 | 17158 | 28659 | 20380 |
| PC3 | 14902 | 16218 | 1699 | 13666 | 29622 | 20872 |
| HELA | 32021 | 34040 | 1418 | 15962 | 33338 | 36371 |
| Caki2 | 7692 | 91219 | 1494 | 10381 | 18825 | 53870 |
| 786O | 26449 | 99063 | 1487 | 19024 | 31310 | 37510 |
| A375 | 17504 | 30583 | 2379 | 10226 | 13748 | 15661 |
| NCIH226 | 5495 | 68710 | 3729 | 2373 | 7042 | 12122 |
| NCIH460 | 15136 | 37452 | 1791 | 10080 | 18243 | 18752 |
| NCIH810 | 14805 | 24670 | 1170 | 6683 | 12546 | 17568 |
| NCIH1838 | 7356 | 12274 | 929 | 5222 | 11945 | 14544 |
| NCIH2030 | 14020 | 17601 | 768 | 9569 | 19200 | 14457 |
| NCIH358 | 2502 | 11214 | 2965 | 2668 | 3651 | 9326 |
| NCIH1793 | 6456 | 33713 | 564 | 3473 | 8382 | 14439 |
| NCIH2347 | 10672 | 36595 | 2670 | 9197 | 13434 | 15359 |
| NCIH1975 | 7841 | 18737 | 1055 | 3644 | 7567 | 14079 |
| A549 | 27484 | 71674 | 2117 | 14901 | 28781 | 27668 |
| NCIH23 | 684 | 23491 | 3220 | 560 | 652 | 13043 |
| NCIH2228 | 5687 | 33141 | 1758 | 2147 | 2899 | 15074 |
| NCIH661 | 9237 | 22777 | 847 | 5371 | 11217 | 12406 |
| NCIH1703 | 19185 | 47070 | 1016 | 9976 | 27116 | 12854 |
| NCIH1581 | 11933 | 237 | 1092 | 4047 | 11377 | 17791 |
| NCIH1563 | 6563 | 56499 | 520 | 1627 | 3645 | 14726 |
| NCIH522 | 6326 | 2517 | 1574 | 5067 | 5606 | 15596 |
| NCIH1437 | 20575 | 34322 | 1506 | 7952 | 7466 | 13929 |
| NCIH441 | 10804 | 2698 | 1320 | 7427 | 11949 | 17031 |
| NCIH69 | 11820 | 5823 | 493 | 4486 | 8939 | 22812 |
| NCIH1048 | 12581 | 55503 | 710 | 8813 | 19372 | 27484 |
| NCIH2170 | 8374 | 1405 | 578 | 7187 | 9461 | 19383 |
| SKMES1 | 14253 | 110152 | 2088 | 8000 | 18861 | 14543 |
| CHAGOK1 | 13970 | 16762 | 881 | 6436 | 15806 | 13886 |

[symbol]

indicates data missing or illegible when filed

### EXAMPLE

TABLE 5
Results of the correlation analyses

| Gene | All cell lines | All lung cell lines | Breast cell lines |
|---|---|---|---|
| CCNE2 | no correlation | no correlation | r = 0.79 |
| | | | P = 0.0003 |
| | | | highly significant |
| MAD2L2 | no correlation | no correlation | r = 0.74 |
| | | | P = 0.00097 |
| | | | highly significant |

The sensitivity of 51 human tumour cell lines with respect to Compound A was determined in proliferation assays. The 1050 values determined were correlated to the relative mRNA amounts of 62 cell cycle-regulatory proteins determined in independent gene chip hybridization studies (Affymetrix technology). Genes, for which statistically significant correlations (P<0.05) were found within the breast tumour cell lines are summarized in Table 5. The correlation coefficients and significance values were calculated using Microsoft Excel 2003 and SigmaStat 3.0.

Viewed across all cell lines analyzed, and in the partial groups of the lung cell lines, there is no correlation between the mRNA amount of the genes CCNE2 (cyclin E2) or MAD2L2 and the 1050 of the cell lines for Compound A. Surprisingly, for the partial group of the 16 breast tumour cell lines, correlation analysis shows a statistically highly significant correlation of the mRNA amount of the genes CCNE2 or MAD2L2 with the sensitivity of the cells, determined as 1050, for Compound A (Tab. 5.).

These data confirm that the relative mRNA amounts of the genes CCNE2 and/or MAD2L2 may indicate the sensitivity of human breast tumour cells for Compound A. A high relative mRNA amount of the genes CCNE2 and/or MAD2L2, for which a positive correlation coefficient was found, shows a higher 1050, equivalent with a lower sensitivity of the cells for Compound A.

### FIGURES

FIG. 1. Schematic representation of the sensitivity of the human breast tumour cell lines with respect to Compound A determined as IC 50 [nM] in proliferation assays against the relative mRNA amount of the gene MAD2L2. The solid line represents the correlation line.

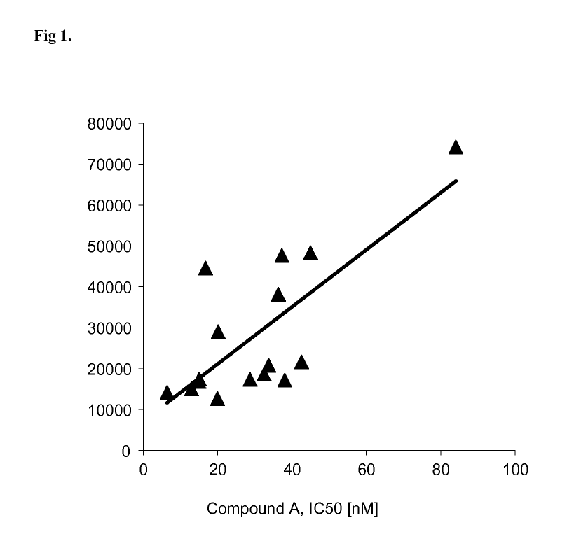

1. Use of MAD2L2 as stratification marker in the treatment of breast tumours with a compound of the general formula (I) 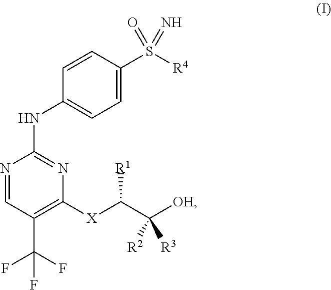
in which
X represents —O— or —NH—, and
R1 represents a methyl, ethyl, propyl or isopropyl group, and
R2 and R3 independently of one another represent hydrogen, a methyl or ethyl group, and
R4 represents a C1-C6-alkyl group or a C3-C7-cycloalkyl ring,
or with one of its physiologically acceptable salts, diastereomers or enantiomers.

2. Use according to claim 1 in the treatment with a compound of the general formula (I) in which
X represents —O— or —NH—, and
R1 represents a methyl group, and
R2 represents a methyl group, and
R3 represents hydrogen or a methyl group, and
R4 represents a methyl or ethyl group or represents a cyclopropyl ring,
or with one of its physiologically acceptable salts, diastereomers or enantiomers.

3. Use according to claim 1 in the treatment with (2R,3R)-3-{[2-{[4-(R-cyclopropylsulphonimidoyl)phenyl]amino}-5-(trifluoromethyl)pyrimidin-4-yl]oxy}butan-2-ol.

4. Use according to claim 1 in the treatment of breast tumours in monotherapy or in combination therapy.

5. Method for the selection of breast tumour patients who may respond to treatment with a compound of the formula (I), characterized in that the extent of expression of MAD2L2 is determined.

6. Method according to claim 5, characterized in that the extent of expression of MAD2L2 is determined at the nucleic acid level.

7. Method according to claim 5, characterized in that the extent of expression of MAD2L2 is determined at the protein level.

8. Method according to claim 5, characterized in that the extent of expression of MAD2L2 is determined on a sample from the cell culture.

9. Method according to claim 5, characterized in that the extent of expression of MAD2L2 is determined on a sample from a mammalian organism.

10. Method according to claim 5, characterized in that the extent of expression of MAD2L2 is determined on a sample from a human patient.

11. Method according to claim 5, characterized in that the extent of expression of MAD2L2 is determined on a sample from a culture of cells or from an experimental animal.
